(19) United States
(12) Patent Application Publication
(10) Pub. No.: US 2008/0114375 A1
(43) Pub. Date: May 15, 2008

(54) METHOD AND APPARATUS FOR ATTACHING A DYNAMIC REFERENCE

(75) Inventor: Ron Andrew von Jako, Melrose, MA (US)

Correspondence Address: PETER VOGEL;GE HEALTHCARE, 3000 N. GRANDVIEW BLVD., SN-477, WAUKESHA, WI, 53188, US

(73) Assignee: General Electric Company, Schenectady, NY (US)

(21) Appl. No.: 11/558,000

(22) Filed: Nov. 9, 2006

(51) Int. Cl. A61B 19/00

(52) U.S. Cl. 606/130

(57) ABSTRACT

A reference platform adapted to facilitate the attachment of a reference unit to a patient is disclosed herein. The reference platform includes a top surface adapted to contact a reference unit, and a bottom surface generally opposite the top surface. The bottom surface is adapted to contact a patient. The reference platform also includes a spike extending in a direction away from the bottom surface. The spike is configured to penetrate the patient in order to secure the reference platform to the patient. A corresponding method for mounting the reference unit to the patient is also provided.

### FIELD OF THE INVENTION

This disclosure relates generally to a method and apparatus for rigidly attaching a dynamic reference to a patient.

### BACKGROUND OF THE INVENTION

In minimally invasive surgical procedures, access to the body is obtained through one or more natural openings or small percutaneous incisions. Medical devices are inserted through these openings and directed to a region of interest within the body. Direction of the medical devices through the body is facilitated by navigation technology wherein the location of a medical device is measured and virtually superimposed on an image of the region of interest. The image may be a pre-acquired image, or an image obtained in near real-time or real-time using known imaging technology such as X-ray, computed tomography (CT), magnetic resonance (MR), or ultrasound (US).

Conventional navigation technology measures the position and orientation of a remote unit attached to the medical device relative to a reference unit. Patient motion can be taken into account by mounting the reference unit directly onto the patient. A reference unit attached in this manner is also referred to as a dynamic reference because it moves along with the patient.

Relative motion between the dynamic reference and the patient can introduce imprecision into a navigation system. It is therefore important that the dynamic reference be securely and rigidly mounted to the patient while also being minimally invasive. A problem with conventional dynamic reference devices relates to their size, high-profile designs and various methods of secure anatomical attachment. Conventional dynamic reference devices are large enough to potentially limit surgical access, their high-profile design poses an increased risk of unintentional contact or bumping, and some mechanical designs are suboptimal for secure anatomical fixation given the anatomical point of interest and procedure at hand.

### BRIEF DESCRIPTION OF THE INVENTION

The above-mentioned shortcomings, disadvantages and problems are addressed herein which will be understood by reading and understanding the following specification.

In an embodiment, a reference platform includes a top surface adapted to contact a reference unit, and a bottom surface generally opposite the top surface. The bottom surface is adapted to contact a patient. The reference platform also includes a spike extending in a direction away from the bottom surface. The spike is configured to penetrate the patient in order to secure the reference platform to the patient. The reference platform is adapted facilitate the attachment of the reference unit to the patient.

In another embodiment, a navigation system includes a reference platform attachable to a patient. The reference platform includes a top surface and a bottom surface generally opposite the top surface. The bottom surface is adapted to contact the patient. The reference platform also includes a spike extending in a direction away from the bottom surface. The spike is configured to penetrate the patient in order to secure the reference platform to the patient. The navigation system also includes a reference unit mountable to the top surface of the reference platform, and a process in communication with the reference unit. The process is adapted to determine the position and/or the orientation of a medical device relative to the reference unit.

In yet another embodiment, a method for mounting a reference unit of a navigation system to a patient includes providing a reference platform having a spike, and attaching the reference platform to the patient such that the spike penetrates a bone. The method also includes attaching a reference unit to the reference platform, and applying a retention device adapted to prevent the reference platform from inadvertently pulling out of the patient.

In yet another embodiment, an anchor device includes a body portion, and a clamp portion attached to the body portion. The clamp portion includes a first leg, and a second leg attached to the first leg such that the first and second legs define a gap therebetween through which the wire is passed. The clamp portion also includes an adjustable member configured to selectively draw the first and second legs together such that a clamping force is applied to the wire. The anchor device is configured to interrupt the transfer of a force applied to a first portion of the wire such that a device connected to a second portion of the wire is not disturbed by the force.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a navigation system in accordance with an embodiment;

FIG. 2 is a perspective illustration of a reference platform;

FIG. 3 is a side sectional view of the reference platform of FIG. 2;

FIG. 4 is a side sectional view of a reference unit of FIG. 1 and a reference platform of FIG. 2 attached to a patient;

FIG. 5 is a perspective view of a blunt impactor in accordance with an embodiment; and

FIG. 6 is a perspective view of a wire anchor in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

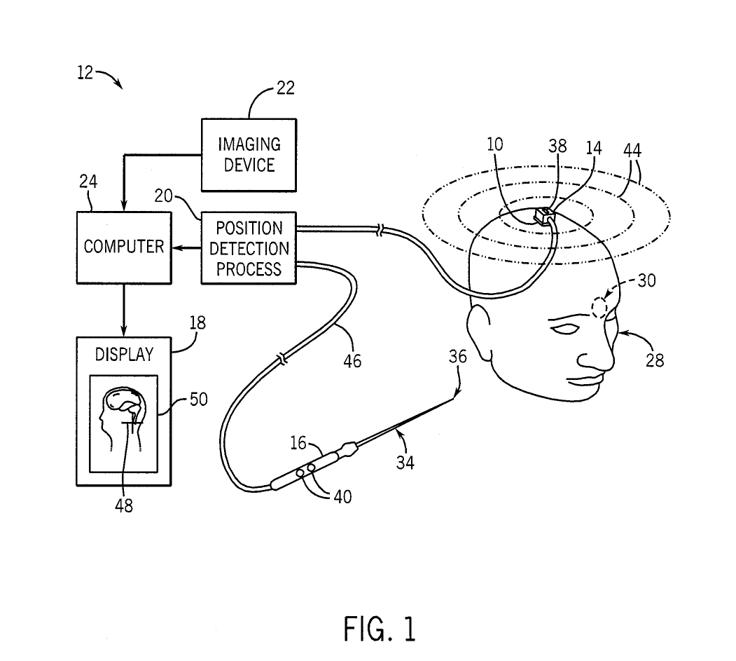

Referring to FIG. 1, a reference platform 10 is shown. It should be appreciated that the reference platform 10 is shown for exemplary purposes and that other configurations may be envisioned. The reference platform 10 may be implemented with a variety of different navigation systems such as, for example, the surgical navigation system 12. The navigation system 12 includes a reference unit 14, a remote unit 16, a display 18, a position detection process 20, an imaging device 22 and a computer 24.

The reference platform 10 is configured to facilitate the rigid attachment of the reference unit 14 to a patient 28 near the target operation site 30 (e.g., the pituitary gland or a frontal trephination procedure, etc) as will be described in detail hereinafter. A reference unit attached in this manner is also referred to as a “dynamic reference” because it moves along with the patient. The remote unit 16 is attached to a medical device 34. The present invention will hereinafter be described in accordance with an embodiment wherein the reference unit 14 includes a field generator 38, and the remote unit 16 includes one or more field sensors 40. It should, however, be appreciated that according to alternate embodiments the reference unit may include the field sensors and the remote unit may include the field generator.

The field generator 38 in the reference unit 14 generates a position characteristic field 44 in an area that includes the target operation site 30. The field sensors 40 in the remote unit 16 produce sensor signals (not shown) in response to the sensed position characteristic field 44. The sensor signals are transmitted or input into the position detection process 20. The sensor signals may be transmitted via communication line or wire 46, or may be wirelessly transmitted. The position detection process 20 is adapted to determine the location of the remote unit 16 relative to the reference unit 14. A known calibration procedure can be implemented to estimate the location of the distal end or tip 36 of the medical device 34.

The location of the medical device 34 may be conveyed via the display 18. According to a preferred embodiment, a graphical representation 48 of the distal end 36 is virtually superimposed onto a patient image 50. More precisely, the graphical representation 48 of the distal end 36 is virtually superimposed onto the portion of the image 50 that corresponds to the actual location of the distal end 36 within the patient 28. The graphical representation 48 may include a dot or cross hairs identifying just the distal end 36, or may include a more complete rendering showing the medical device 34 in detail.

According to one embodiment, the patient image 50 is obtained prior to the medical procedure using known imaging technology such as X-ray, computed tomography (CT), magnetic resonance imaging (MRI), or ultrasound (US). Additionally, during the course of the medical procedure, the imaging device 22 may be implemented to observe the patient 28 in real-time or near real-time. Therefore, the pre-recorded patient image 50 can be replaced with a real-time patient image or a near real-time image as desired. According to an exemplary embodiment, the imaging device 22 may include a fluoroscopic X-ray device mounted to a C-arm, however, other imaging devices may also be implemented.

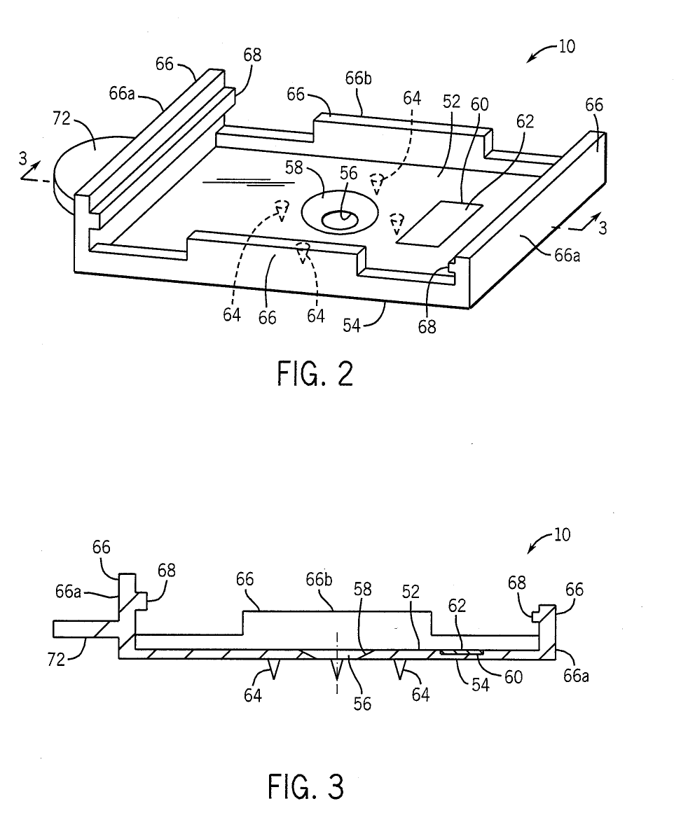

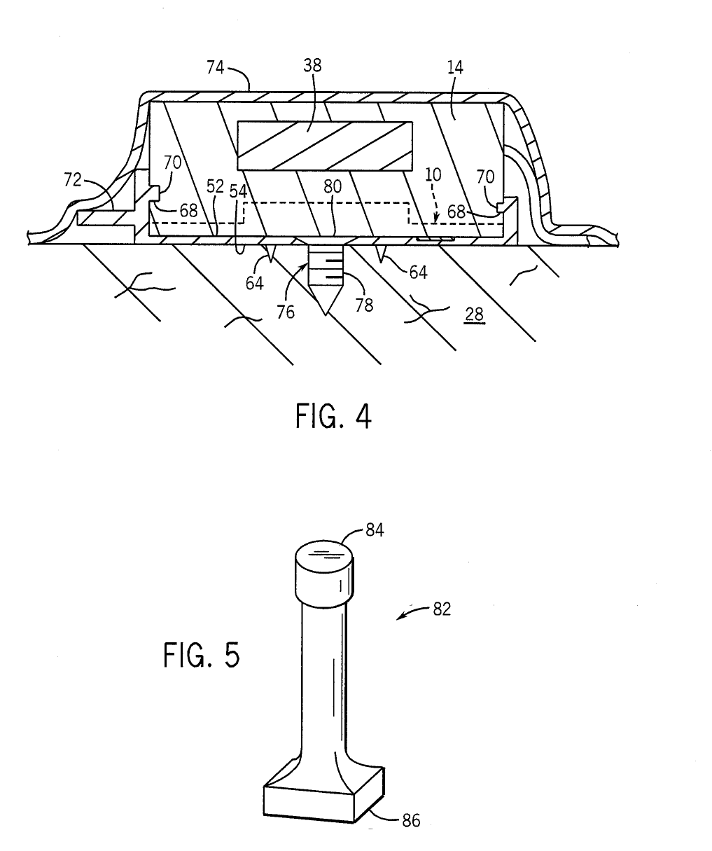

FIGS. 2 and 3 show the reference platform 10 in more detail. More precisely, FIG. 2 is a perspective view of the reference platform 10, and FIG. 3 is a sectional side view of the reference platform 10. The reference platform 10 is generally rectangular and is sized to receive the reference unit 14 (shown in FIG. 4). The reference platform 10 may comprise relatively inexpensive plastic materials (e.g., a medical grade radiolucent plastic), and can be produced in accordance with inexpensive manufacturing processes (e.g., injection molding) in order to provide a disposable reference platform 10. Advantageously, providing a disposable reference platform 10 minimizes surgical instrument counts and reprocessing of small instruments.

The reference platform 10 includes a top surface 52, a bottom surface 54, and a screw hole 56 defined therebetween. The top surface 52 is configured to engage or contact the reference unit 14 (shown in FIG. 4), and the bottom surface 54 is configured to engage or contact the patient 28 (shown in FIG. 1). The top surface 52 includes a counter-sunk portion 58 formed around the periphery of the screw hole 56 such that the head 80 of a cranial screw 76 (shown in FIG. 4) inserted through the screw hole 56 remains generally flush with the top surface 52. According to an embodiment, the top surface 52 defines a recessed portion 60 adapted to receive a temperature sensor 62 that is schematically depicted in FIGS. 2 and 3. The recessed portion 60 is adapted to retain the temperature sensor 62 such that the temperature sensor 62 remains generally flush with the top surface 52. By positioning the temperature sensor 62 in this manner, the temperature sensor 62 can engage and thereby accurately monitor the temperature of the reference unit 14 in a manner that does not impede the attachment of the reference unit 14 to the reference platform 10. The temperature sensor 62 may therefore be implemented to monitor the temperature of the reference unit 14 in order to prevent the accumulation of heat from the field generator 38 (shown in FIG. 4).

A plurality of spikes 64 extend from the bottom-surface 54. According to one embodiment, the reference platform 10 includes four evenly spaced spikes 64 comprised of a non-ferrous material in order to avoid distorting the position characteristic field 44 (shown in FIG. 1). The spikes 64 are available in a variety of different lengths and thicknesses to accommodate different hair, soft tissue and bone depths/densities. The spikes 64 are intended to penetrate the skin and the cranium without significant purchase penetration and without absolute penetration of the bony cranium such that the reference platform 10 is rigidly mounted to the patient 28 (shown in FIG. 1) in a minimally invasive manner. As will be described in detail hereinafter, when the spikes 64 are engaged, they are configured to limit the translation of the reference platform 10 relative to the patient 28. The spikes 64 are not, however, designed to resist disengagement (or pull-out) from the patient 28.

The reference platform 10 includes a plurality of walls 66 adapted to secure the reference unit 14 (shown in FIG. 4), and to resist the rotation of the reference unit 14 relative to the reference platform 10. The walls 66 are low-profile meaning they protrude from the top surface 52 by a small amount. The low-profile walls 66 minimize the risk of unintentional contact or bumping which could shift the reference platform 10 relative to the patient 28 (shown in FIG. 1) and thereby introduce imprecision into the navigation system 12 (shown in FIG. 1). According to one embodiment, the walls 66 include a first pair of generally parallel walls 66a, and a second pair of generally parallel walls 66b. The walls 66a each include an attachment feature such as the tab 68. The tabs 68 are configured to engage complementary grooves 70 (shown in FIG. 4) defined in the reference unit 14. Therefore, as the reference unit 14 is brought into contact with the top surface 52 of the reference platform 10, each tab 68 is introduced into a corresponding groove 70 such that the reference unit 14 is mechanically retained by the reference platform 10. It should be appreciated that the previously described mechanical retention structure is described for exemplary purposes, and that the reference unit 14 may alternatively be attached to the reference platform 10 in any known manner.

The reference platform 10 includes a lip 72 extending in an outward direction from one of the walls 66a. The lip 72 is configured to facilitate the removal of the reference unit 14 (shown in FIG. 4) from the reference platform 10. When the reference unit 14 is attached to the reference platform 10, the lip 72 protrudes away from the body of the reference unit 14 such that a user can easily engage or push on the lip 72 without also engaging the reference unit 14. Therefore, an individual can push down on or pinch the lip 72 in order hold the reference platform 10 such that when the reference unit 14 is removed, it separates from the reference platform 10 without also removing the reference platform 10. The reference platform 10 is also designed so that pushing on the lip 72 applies both a downward force to hold the reference platform 10 in place and also a moment of inertia tending to pull the attached wall 66a away from the reference unit 14. The act of pulling the attached wall 66a away from the reference unit 14 also de-couples the notch 68 from the groove 70 such that the reference unit 14 is mechanically released from the reference platform 10 and the two components can be easily separated.

Referring to FIG. 4, a sectional side view shows the reference unit 14 and the reference platform 10 attached to the patient 28. As previously indicated, the spikes 64 are configured to limit the translation of the reference platform 10 relative to the patient 28, but the spikes 64 are not designed to resist disengagement (or pull-out). Therefore, the spikes 64 are generally implemented in combination with one or more additional retention devices such as the tape 74 and the screw 76 in order to ensure the reference platform 10 is not inadvertently pulled-out of the patient 28. The tape 74 may include a sterile surgical tape, and may be applied as an oversized patch or as a smaller strip. The sterile surgical adhesive tape 74 is designed such that it is specific to this reference platform and to provide secure fixation to all independent regions of the cranium, similar to a head-band in some cases. The surgical tape 74 is also single per patient use. The screw 76 includes a screw body 78 and a screw head 80. The screw 76 may include a cranial bone screw that is generally implemented for applications requiring stronger fixation without the need of a surgical adhesive tape.

There are several methods for attaching the reference platform 10 to the patient 28. A first method includes manually pushing the reference platform 10 such that the spikes 64 pierce the patient's cranium. This first method may be performed, for example, with the user's thumb in a manner similar to that of a thumbtack.

A second method for attaching the reference platform 10 to the patient 28 includes implementing a tool such as the impactor 82 shown in FIG. 5. The impactor 82 includes a first end 84 that is struck or impacted, and a second end 86 adapted to engage the reference platform 10 (shown in FIG. 2). The first end 84 may be struck with a conventional device such as a rubber reflex hammer (not shown). The second end 86 is in contact with the reference platform 10 such that the force applied by the reflex hammer is transferred through the impactor 82 in order to drive the spikes 64 (shown in FIG. 4) into the patient's surface cranium. According to one embodiment, the second end of the impactor 82 generally conforms to the geometry of the top surface 52 of the reference platform 10. This conformal geometry has the effect of more evenly distributing the force applied by the reflex hammer to each of the spikes 64, which improves the likelihood that the spikes 64 will all fully engage the patient 28.

A third method for attaching the reference platform 10 to the patient 28 (shown in FIG. 1) includes implementing an applicator gun (not shown) that functions similarly to a staple gun. The applicator gun is loaded with the reference platform 10 as a single cartridge. The applicator gun press fits the reference platform 10 into the cranium with enough force to enable to spikes 64 to grab the surface of the bone.

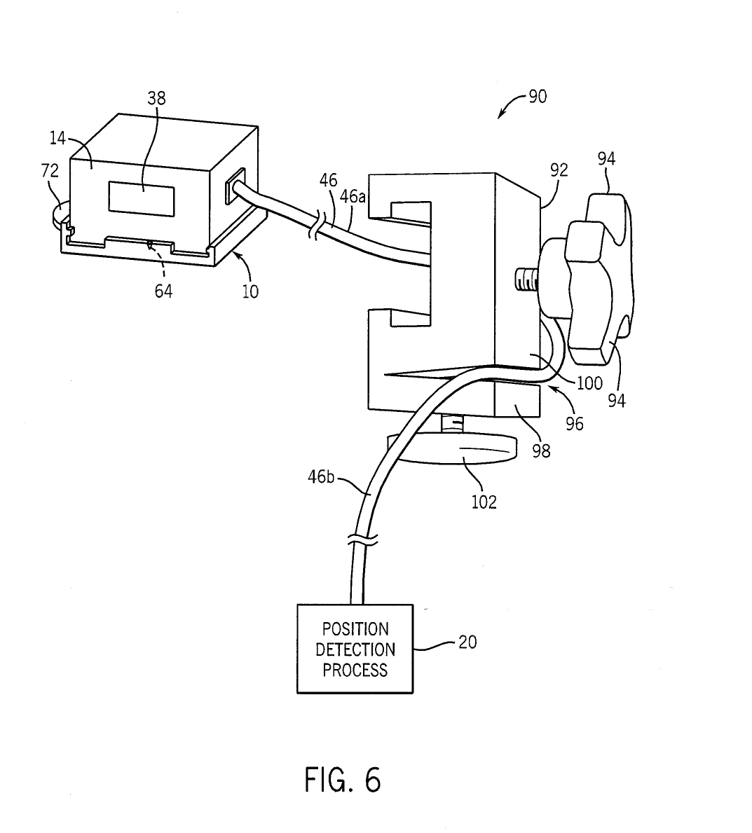

Referring to FIG. 6, the reference unit 14 and reference platform 10 may be implemented with a wire anchor 90. The wire anchor 90 is adapted to secure the reference unit wire 46 and thereby minimize any risk associated with unintentionally pulling on the wire 46.

The wire anchor 90 includes a generally C-shaped body portion 92 adapted for attachment to a bed rail (not shown). A first threaded member 94 is rotatable to control the degree of insertion through a wall of the C-shaped body portion 92. When the C-shaped body portion 92 is mounted to a bed rail, the first threaded member 94 can be rotated in a first direction in order to engage the bed rail and thereby tighten the attachment of the wire anchor 90. Conversely, the first threaded member 94 can be rotated in a second direction in order to disengage the bed rail and thereby loosen the attachment of the wire anchor 90.

The wire anchor 90 also includes a radiolucent clamp portion 96 configured to secure the reference unit wire 46. The clamp portion 96 includes a first leg 98 and a second leg 100 that together generally form a V-shape. A second threaded member 102 passes through the first leg 98 and into the second leg 100. The second threaded member 102 can be rotated in a first direction to apply a compressive force tending to draw the first and second legs 98, 100 together. Conversely, the second threaded member 102 can be rotated in a second direction to release the compressive force such that the first and second legs 98, 100 separate until they reach their steady state positions.

A method for implementing the wire anchor 90 includes the following. The C-shaped body portion 92 of the wire anchor 90 is mounted to a bed rail (not shown), and the first threaded member 94 is rotated so that the wire anchor 90 becomes tightly affixed to the bed rail. The reference unit wire 46 is then passed through the clamp portion 96 such that a first wire section 46a is defined between the reference unit 14 and the wire anchor 90, and a second wire section 46b is defined between the wire anchor 90 and a device incorporating the position detection process 20. Thereafter, the second threaded member 102 is rotated in a direction adapted to draw the first and second legs 98, 100 together such that the first and second legs 98, 100 apply a compressive force to the reference wire 46 disposed therebetween. It can be seen that by securing the reference unit wire 46 in the manner described, a force applied to the second wire section 46b is prevented from reaching the reference unit 14. Therefore, if the second wire section 46b is inadvertently pulled, the reference unit 14 and patient 28 (shown in FIG. 1) are unaffected.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A reference platform comprising:
a top surface adapted to contact a reference unit;
a bottom surface generally opposite the top surface, said bottom surface adapted to contact a patient; and
a spike extending in a direction away from the bottom surface, said spike being configured to penetrate the patient in order to secure the reference platform to the patient;
wherein the reference platform is adapted facilitate the attachment of the reference unit to the patient.

2. The reference platform of claim 1, further comprising a wall extending in a direction away from the top surface.

3. The reference platform of claim 2, further comprising a screw hole defined between the top surface and the bottom surface.

4. The reference platform of claim 3, further comprising a screw at least partially disposed within the screw hole.

5. The reference platform of claim 4, wherein said top surface defines a counter sunk portion around the periphery of the screw hole, said counter sunk portion adapted to accommodate a head portion of the screw.

6. The reference platform of claim 1, wherein said top surface defines a recessed portion adapted to retain a sensor.

7. The reference platform of claim 2, wherein said wall is a low-profile wall.

8. The reference platform of claim 2, wherein said wall includes a tab adapted to engage the reference unit such that the reference unit is mechanically retained by the reference platform.

9. The reference platform of claim 2, further comprising a lip extending in an outward direction from the wall.

10. A navigation system comprising:
a reference platform attachable to a patient, said reference platform comprising:
a top surface;
a bottom surface generally opposite the top surface, said bottom surface adapted to contact the patient; and
a spike extending in a direction away from the bottom surface, said spike being configured to penetrate the patient in order to secure the reference platform to the patient;
a reference unit mountable to the top surface of the reference platform; and
a process in communication with the reference unit, said process adapted to determine the position and/or the orientation of a medical device relative to the reference unit.

11. The navigation system of claim 10, further comprising a retention device applied to the patient in order to prevent the reference platform from unintentionally pulling out.

12. The navigation system of claim 11, wherein the retention device includes surgical tape.

13. The navigation system of claim 11, wherein the retention device includes a screw.

14. The navigation system of claim 10, wherein the remote device is configured to wirelessly transmit data to the process.

15. The navigation system of claim 10, wherein the remote device includes a wire adapted to transmit data to the process.

16. The navigation system of claim 15, further comprising a wire anchor device attached to the wire.

17. A method for mounting a reference unit of a navigation system to a patient comprising:
providing a reference platform having a spike;
attaching the reference platform to the patient such that the spike penetrates a bone;
attaching a reference unit to the reference platform; and
applying a retention device adapted to prevent the reference platform from inadvertently pulling out of the patient.

18. The method of claim 17, wherein said attaching the reference platform to the patient includes manually pushing the reference platform into engagement with the patient such that the spike is manually driven into the bone.

19. The method of claim 17, wherein said attaching the reference platform to the patient includes striking a device configured to distribute the applied force across a surface of the reference platform such that spike is driven into the bone.

20. The method of claim 17, wherein said attaching the reference platform to the patient includes implementing an attachment gun to drive the spike into the bone.

21. The method of claim 17, wherein said applying a retention device includes taping the reference platform to the patient.

22. The method of claim 17, wherein said applying a retention device includes screwing the reference platform to the patient.

23. An anchor device comprising:
a body portion;
a clamp portion attached to the body portion, said clamp portion including:
a first leg;
a second leg attached to the first leg, said first and second legs defining a gap therebetween through which the wire is passed;
an adjustable member configured to selectively draw the first and second legs together such that a clamping force is applied to the wire;
wherein the anchor device is configured to interrupt the transfer of a force applied to a first portion of the wire such that a device connected to a second portion of the wire is not disturbed by the force.

24. The anchor device of claim 23, wherein the body portion is attachable to a bed rail.

25. The anchor device of claim 24, further comprising a second adjustable member adapted to control the tightness with which the body portion is attached to the bed rail.
